(19) United States
(12) Patent Application Publication
(10) Pub. No.: US 2022/0047374 A1
(43) Pub. Date: Feb. 17, 2022

(54) STENT GRAFT SYSTEMS AND METHODS WITH INFLATABLE FILL STRUCTURE AND FILLABLE CUFF

(71) Applicant: Endologix LLC, Irvine, CA (US)

(72) Inventors: Christopher Williams, Santa Rosa, CA (US); Rajesh Radhakrishnan, Rancho Santa Margarita, CA (US); Kaushik Patel, Poway, CA (US)

(21) Appl. No.: 17/274,754

(22) PCT Filed: Sep. 11, 2019
(86) PCT No.: PCT/US2019/050684

§ 371 Date: Mar. 9, 2021

Related U.S. Application Data
(60) Provisional application No. 62/730,441, filed on Sep. 12, 2018.

(51) Int. Cl. A61F 2/07

(52) U.S. Cl. CPC A61F 2/07; A61F 2250/0003; A61F 2002/067; A61F 2002/075; A61F 2002/077

(57) ABSTRACT

A stent graft system includes a stent graft an inflatable fill structure, and a cuff. The inflatable fill structure at least partially surrounds the stent graft. In various arrangements, the inflatable fill structure has a cavity that is bifurcated. A portion of the cavity is configured to receive a branch stent graft for connection to the stent graft. The cuff is fillable and is located outside of the inflatable fill structure, and allows for providing a seal with a wall of a blood vessel. The cuff and the inflatable fill structure are separately fillable from each other to different pressures with fill medium. In various arrangements, the cuff has a tapered shape such that it is wider at one end than at an opposite end when filled with a fill medium. A method includes filling the cuff to a higher pressure than a pressure of the inflatable fill structure.

### CROSS-REFERENCE TO RELATED PATENT APPLICATIONS

This application claims priority from U.S. Provisional Patent App. Ser. No. 62/730,441, filed Sep. 12, 2018, the entire contents of which are incorporated by reference herein.

### FIELD

The present technology relates generally to endoluminal vascular prostheses and methods of placing such prostheses. More particularly, various arrangements relate to stent graft systems and to methods of placing such stent graft systems for treating aortic aneurysms.

### BACKGROUND

Aneurysms are enlargements or bulges in blood vessels that are often prone to rupture and which therefore present a serious risk to a patient. Aneurysms may occur in any blood vessel but are of particular concern when they occur in the cerebral vasculature or the patient's aorta.

Abdominal aortic aneurysms (AAA's) are classified based on their location within the aorta as well as their shape and complexity. Aneurysms that are found below the renal arteries are referred to as infrarenal abdominal aortic aneurysms. Suprarenal abdominal aortic aneurysms occur above the renal arteries. Thoracic aortic aneurysms (TAA's) occur in the ascending, transverse, or descending part of the upper aorta. Infrarenal aneurysms are the most common, representing about 70% of all aortic aneurysms. Suprarenal aneurysms are less common, representing about 20% of the aortic aneurysms. Thoracic aortic aneurysms are the least common and often the most difficult to treat.

The most common form of aneurysm is “fusiform,” where the enlargement extends about the entire aortic circumference. Less commonly, the aneurysms may be characterized by a bulge on one side of the blood vessel attached at a narrow neck. Thoracic aortic aneurysms are often dissecting aneurysms caused by hemorrhagic separation in the aortic wall, usually within the medial layer. A common treatment for each of these types and forms of aneurysm is open surgical repair. Open surgical repair is quite successful in patients who are otherwise reasonably healthy and free from significant co-morbidities. Such open surgical procedures are problematic, however, since access to the abdominal and thoracic aortas is difficult to obtain and because the aorta must be clamped off, placing significant strain on the patient's heart.

Endoluminal grafts have come into widespread use for the treatment of aortic aneurysms in patients. A typical endograft procedure utilizes a stent graft placement to treat the aneurysm. The purpose of the graft is generally to isolate the diseased portion of the aortic wall from the aortic blood pressure and prevent further dilatation or rupture of the diseased portion of the aortic wall. In general, endoluminal repairs access the aneurysm “endoluminally” through either or both iliac arteries. The grafts are then implanted. Successful endoluminal procedures have a much shorter recovery period than open surgical procedures.

### SUMMARY OF THE DISCLOSURE

Various stent graft systems and methods described herein are directed to treating aneurysms. Various arrangements allow for improved sealing and anchoring of a stent graft system. A stent graft system in accordance with various arrangements includes a stent graft, an inflatable fill structure, and a cuff. The inflatable fill structure at least partially surrounds the stent graft. In some arrangements, the inflatable fill structure has a cavity that is bifurcated. The cuff is fillable and is located outside of the inflatable fill structure.

In various arrangements, a portion of the cavity is configured to receive a branch stent graft for connection to the stent graft. In various arrangements, the cuff is located at an end of the stent graft. In some arrangements, the cuff has a tapered shape such that it is wider at one end than at an opposite end when filled with a fill medium. In some arrangements, the stent graft system further includes inflatable channels located at least partially around the stent graft and surrounded by the inflatable fill structure. In various arrangements, the cuff and the inflatable fill structure are separately fillable from each other to different pressures with fill medium.

In various arrangements, the inflatable fill structure is configured such that the cavity that is bifurcated is longer on one side of the bifurcation than on the other side of the bifurcation. In some arrangements, a scaffold mechanism is located in the cavity at least partially in an area where a branch stent graft is insertable into the cavity, and the scaffold mechanism is configured to provide structural support to the inflatable fill structure prior to the branch stent graft being received within the cavity. Also, in some arrangements, the stent graft system further includes a longitudinal support structure anchored to an end of the inflatable fill structure and structurally coupled to the scaffold mechanism. In various arrangements, the stent graft includes a main stent graft and a branch stent graft that are integrally formed, and the main stent graft includes a cavity for receiving a second branch stent graft.

A method in accordance with various aspects provides for using a stent graft system that includes a stent graft, an inflatable fill structure, and a cuff. The method includes filling the inflatable fill structure that at least partially surrounds the stent graft and that has a cavity that is bifurcated, and filling the cuff that is located outside of the inflatable fill structure to form a seal with a wall of a blood vessel. In various aspects, the method further includes inserting a branch stent graft into the cavity of the inflatable fill structure. Also, in various aspects, the method further includes inserting the branch stent graft at least partially into the stent graft.

In various aspects, the cuff and the inflatable fill structure are filled to different pressures. For example, in some aspects, the cuff is filled to a higher pressure than a pressure of filling of the inflatable fill structure. In some aspects, the cuff has a size such that it extends from a bottom of renal arteries to a top of an aneurysm so as to form a seal in an entire proximal neck region of an aorta when filled. In various aspects, the method further includes filling inflatable channels that are located at least partially around the stent graft and that are surrounded by the inflatable fill structure.

In various arrangements, the cuff has a tapered shape such that it is wider at one end than at an opposite end when filled. Also, in some arrangements, one side of the inflatable fill structure that surrounds a first branch stent graft is longer than another side of the inflatable fill structure that surrounds a second branch stent graft. In some aspects, the method further includes structurally supporting a portion of the cavity of the inflatable fill structure with a scaffold mechanism that is separate from the stent graft prior to inserting a branch stent graft into the portion of the cavity. In some arrangements, the stent graft system further includes a longitudinal support structure anchored to an end of the inflatable fill structure and structurally coupled to the scaffold mechanism for structurally supporting the inflatable fill structure.

In various arrangements, the stent graft system comprises a stent graft, an inflatable fill structure at least partially surrounding the stent graft, and a cuff that is fillable and that is located outside of the inflatable fill structure. In some arrangements at least one inflatable fill structure can have at least one cavity. In some arrangements, the inflatable fill structure comprises multiple discrete cavities. In some arrangements, the inflatable fill structure comprises a branched cavity. The inflatable fill structure can comprise cavities configured to provide access to multiple arteries such as iliac and renal arteries. In some arrangements, the inflatable fill structure comprises cavities configured for fluid communication with iliac arteries, renal arteries or both. In some arrangements, the system comprises a fenestrated stent graft. Accordingly, the system can comprise branch stent grafts anchored to iliac and/or renal arteries. In some arrangements, the system comprises a plurality of inflatable fill structures where at least some fill structures are attached to the stent graft. At least some fill structures may be attached to other fill structures.

### BRIEF DESCRIPTION OF THE DRAWINGS

FIG. 1 is a cross-sectional view of an example of a stent graft system deployed across an aneurysm according to various arrangements.

FIG. 2 is a diagram illustrating the stent graft system of FIG. 1 with an inflatable fill structure according to various arrangements.

FIG. 3 is a diagram illustrating a stent graft system with an inflatable fill structure according to various arrangements.

FIG. 4 is a diagram illustrating a stent graft system having a bifurcated inflatable fill structure according to various arrangements.

FIG. 5 is a diagram illustrating the stent graft system of FIG. 4 with a branch stent graft inserted into the bifurcated inflatable fill structure according to various arrangements.

FIG. 6 is a cross-sectional view of an example of a stent graft system deployed across an aneurysm according to various arrangements.

FIG. 7 is a diagram illustrating a stent graft system with an inflatable fill structure according to various arrangements.

FIG. 8 is a cross-sectional view of an example of a stent graft system with a main inflatable fill structure and two branch inflatable fill structures deployed across an aneurysm according to various arrangements.

FIG. 9 is a diagram illustrating a stent graft system having a bifurcated inflatable fill structure according to various arrangements.

FIG. 10 is a cross-sectional view of an example of a stent graft system with a bifurcated inflatable fill structure deployed across an aneurysm according to various arrangements.

FIG. 11 is a diagram illustrating a stent graft system having a bifurcated inflatable fill structure according to various arrangements.

FIG. 12 is a diagram illustrating an inflatable fill structure according to various arrangements.

FIG. 13A illustrates a flowchart of a method of using a stent graft system in accordance with various aspects.

FIGS. 13B, 13C, and 13D illustrate steps that can be used with the method of FIG. 13A in accordance with various aspects.

### DETAILED DESCRIPTION

Various arrangements are described hereinafter. It should be noted that the specific arrangements are not intended as an exhaustive description or as a limitation to the broader aspects discussed herein. One aspect described in conjunction with a particular arrangement is not necessarily limited to that arrangement and may be practiced with any other arrangement(s).

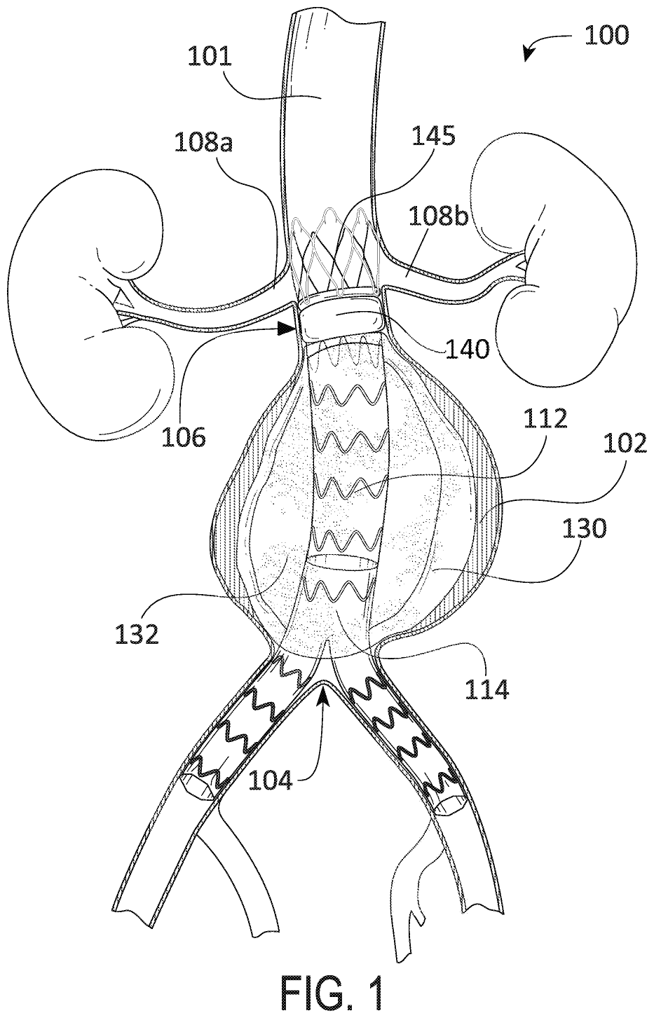

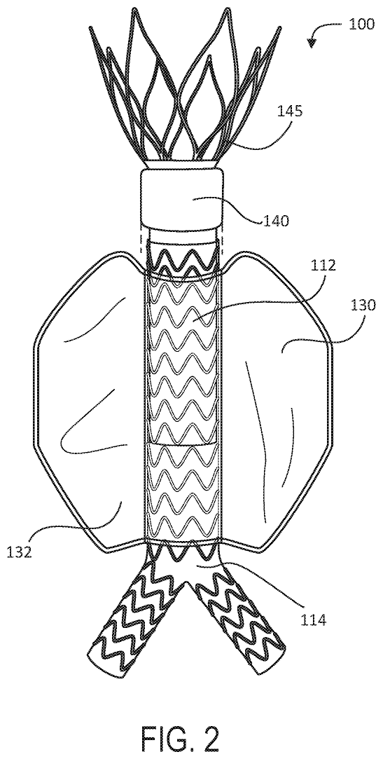

FIG. 1 is a cross-sectional view of an example of a stent graft system 100 deployed across an aneurysm 102 according to various arrangements. FIG. 2 is diagram illustrating the stent graft system 100 with an inflatable fill structure 130 according to various arrangements. Referring to FIGS. 1 and 2, the aneurysm 102 is defined by an aneurysm sac, which is a bulged section of an aorta 101. The aneurysm 102 shown is an infrarenal aortic aneurysm, given that the aneurysm 102 is located below renal arteries 108a and 108b. A segment of the aorta 101 between the renal arteries 108a and 108b and the aneurysm sac is referred to as a proximal neck 106.

The stent graft system 100 includes a first stent graft 112 and a second stent graft 114. In some examples, the second stent graft 114 is a bifurcated stent graft. In some examples, the first stent graft 112 is a proximal extension stent graft. The second stent graft 114 has a proximal end, a distal end, and an outside surface. The second stent graft 114 can be placed onto an aortic bifurcation 104. The aortic bifurcation 104 is the place where the aorta 101 branches into two iliac arteries as shown. The stent graft system 100 includes the inflatable fill structure 130 that at least partially surrounds the first stent graft 112 and the second stent graft 114. The inflatable fill structure 130 is fillable with a fill medium 132. In various examples, the inflatable fill structure 130 is an endobag fixed to a portion of the outside surface of the second stent graft 114 and includes an outer membrane that is configured to extend beyond the proximal end of the second stent graft 114 when the inflatable fill structure 130 is in a filled state in some arrangements. In other arrangements, the outer membrane of the endobag corresponding to the inflatable fill structure 130 does not extend beyond the proximal end of the second stent graft 114.

The stent graft system 100 can be deployed across the aneurysm 102 in any suitable manner. For example, the second stent graft 114 with the inflatable fill structure 130 is first placed onto the aortic bifurcation 104. The inflatable fill structure 130 is initially in an uninflated state. The first stent graft 112 is placed at least partially into a main body of the second stent graft 114. For example, an end of the first stent graft 112 is inserted into the proximal end of the second stent graft 114. In this manner, the first stent graft 112 can extend the aneurysm repair into the proximal neck 106. In various examples, the second stent graft 114 is not bifurcated and can be readily adapted or used in any aneurysm repair using a stent and an inflatable fill structure. In various examples, other types of extension stent grafts can be placed into any luminous opening of the second stent graft 114.

Next, the inflatable fill structure 130 is filled with fill medium 132 to achieve an inflated or filled state. The fill medium 132 pushes a wall of the inflatable fill structure 130 against the aneurysm 102. A portion of the inflatable fill structure 130 extends proximally into a space of the aneurysm 102 adjacent to the first stent graft 112. That is, when in an uninflated state, the inflatable fill structure 130 can be confined to being around the second stent graft 114, but when inflated in the filled state as shown, the inflatable fill structure 130 expands radially and proximally to fill the entire (or most of the) aneurysm 102, including at least a portion of a space around the first stent graft 112 that is not covered by the second stent graft 114. When the inflatable fill structure 130 is filled, the wall of the inflatable fill structure can conform to an inner wall of the aneurysm 102. When the inflatable fill structure 130 is filled, the wall of the inflatable fill structure can conform to at least a portion of an outer surface of the first stent graft 112 and to at least a portion of the outer surface of the second stent graft 114. The inflatable fill structure 130 is configured such that when it is in the filled state, the inflatable fill structure 130 extends beyond the proximal end of the second stent graft 114 and, thus, surrounds at least a portion of the first stent graft 112. In other examples, the inflatable fill structure 130 (in the filled state) only covers the second stent graft 114. In some arrangements, the inflatable fill structure 130 does not cover the bifurcated portion of the second stent graft 114.

In any of the arrangements described herein, the endovascular graft system (e.g., the stent graft system 100) may be fixed at the proximal and/or distal sealing locations (e.g., at the proximal neck 106 and iliac arteries (e.g. at the aortic bifurcation 104) when treating an infrarenal aortic aneurysm. The stent graft system 100 includes additional sealing or anchoring mechanisms including a cuff 140 and a stent-like scaffold structure 145 as shown. In various arrangements, the anchoring mechanisms include but are not limited to stents, scaffolds, hooks, barbs, seals, sealing cuffs, and/or the like. In some arrangements, for sealing cuffs or stents which extend proximately of infrarenal prosthesis, it may be desirable to provide openings or ports to allow the anchoring or sealing devices to extend over the renal ostia while penetrating blood flow into the renal arteries. In various arrangements, the sealing or anchoring devices are attached to and/or overlap with the filling structure of the prosthesis and provide for a smooth transition from the aortic and/or iliac lumens into the tubular lumens provided by the deployed filling structures.

In addition to the filling structures described hereinabove, the graft systems (e.g., the stent graft system 100) may further include at least a first scaffold separate from the filling structure, where the scaffold can be expanded within the generally tubular lumen which provides the blood flow after the filling structure has been deployed in the aneurysm. The first scaffold will be adapted to expand within at least a first portion of the tubular lumen of the filling structure and may provide one or more specific advantages. For example, the scaffold may support and smooth the inside wall of the tubular lumen which in some cases might otherwise become uneven during hardening of the polymer fill. Scaffolds may also provide for anchoring of the filling structure, particularly at the aortic end of the graft when placed in an AAA. The scaffold may be partly or wholly covered with a membrane in order to form a stent-graft. In such cases, the graft structure may help provide a transition from the blood vessel into the generally tubular lumen of the filling structure from the aortic end. Alternatively, the graft structure could provide one or a pair of transitions out of the iliac end of the filling structure. In a particular example, a graft structure can be used on either side of the filling structure in order to treat additional or continuing aneurysmal regions in the adjacent blood vessel. In any arrangements, the system may include multiple scaffold structures. For example, the system may include at least a first and a second scaffold, one for each of the tubular lumens defined by the first and second double-walled filling structures, respectively. The scaffolds may be adapted to be placed in series, frequently overlapping, or may be adapted to be spaced apart at either or both ends and optionally at regions between the ends.

In various arrangements, the stent-like scaffold structure 145 may be implanted in an upper proximal opening of a tubular lumen of a filling structure (e.g., at an edge of the cuff 140) in order to help anchor the upper end of the structure (e.g., the cuff 140 and the first stent graft 112) and prevent intrusion of blood into the region between the outer wall and the inner surface of the aneurysm 102 and to generally improve the transition from the aorta 101 into the tubular lumen. The stent-like scaffold structure 145 may include a stent, graft, and/or other expandable luminal support structure. The first stent graft 112 may include one or more circumferential inflatable channels extending around a circumference of the graft body or that may extend partially around the circumference of the graft body. The circumferential inflatable channels may be in communication with each other via a longitudinal inflatable fill channel. The network of inflatable channels may optionally be filled with a hardenable material that may be configured to harden, cure or otherwise increase in viscosity or become more rigid after being injected into the channels. Hardenable inflation materials such as gels, liquids or other flowable materials that are curable to a more solid or substantially hardened state may be used to provide mechanical support to the graft body by virtue of the mechanical properties of the hardened material disposed within the channels. In some arrangements, the filling agent is saline. In some arrangements, the filling agent is a gas.

The stent graft materials used for the stent graft system 100 include but are not limited to, polyesters, ePTFE, polyurethane, and the like. For example, in some arrangements the cuff 140 has a fill line or a channel used to fill polymers (e.g., polyesters, ePTFE, polyurethane, and the like) in liquid form. The cuff 140 may have a different fill line as compared to the rest of the stent graft system 100 (e.g., the inflatable fill structure 130) in some arrangements. As such, when installing the stent graft system 100, at least a first fill line is used to inject polymer to the inflatable fill structure 130 and a second fill line is used to inject polymer to the cuff 140.

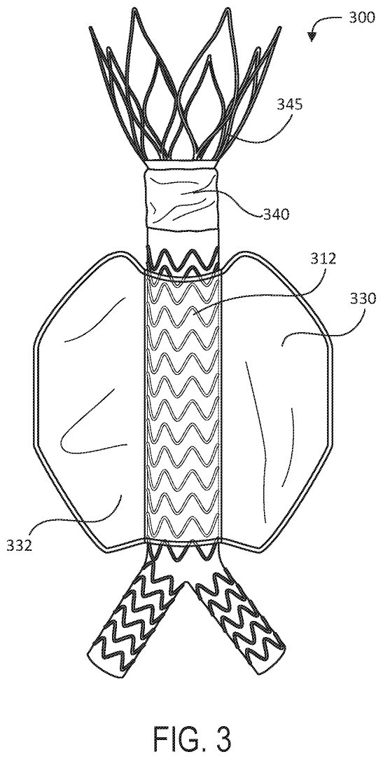

In some arrangements, the cuff 140 is made from a different material as compared to the stent graft material for the rest of the stent graft system 100. In some arrangements, the cuff 140 is made from a same material as the stent graft material for the rest of the stent graft system 100. In some arrangements, the polymer is filled into the cuff 140 via a fill line at a higher pressure as compared to that used to fill the rest of the stent graft system 100 (e.g., the inflatable fill structure 130). For example, the inflatable fill structure 130 can be filled at 0-250 mm Hg, 180-250 mm Hg, 0-100 mm Hg, or 100-250 mm Hg. The cuff 140 can be filled at 180 mm Hg-760 mm Hg (1 atm). The cuff 140 can be filled at a higher pressure because the cuff 140 is contacting healthy tissue, which is capable of handling a higher pressure for sealing and anchoring purposes. The inflatable fill structure 130 on the other hand contacts the aneurysm sac (unhealthy tissue), and therefore should be filled at a lower pressure. A same fill line can be used to fill the cuff 140 and the inflatable fill structure 130 at different pressures, in some examples. In other examples, two different fill lines can be used to fill the cuff 140 and the inflatable fill structure 130 at different pressures, separately. The cuff 140 (e.g., a deflated version of which is displayed as a cuff 340 of FIG. 3) can be inflated, thus forming a unitary structure when the polymers become solidified.

With reference to FIG. 1, the dimensions of the aneurysm 102 can vary greatly from patient to patient. The diameter of the proximal neck 106 may vary, for example, from 18 millimeters (mm) to 34 mm. The distance from the aortic bifurcation 104 to the renal arteries 108a and 108b may vary, for example, from 80 mm to 160 mm. The diameters of the right and left iliac arteries might not be the same. The diameters of the iliac arteries at the aortic bifurcation 104 may vary, for example, from 8 mm to 20 mm. One iliac artery or both iliac arteries may be aneurysmal with greatly enlarged diameters, for example, of more than 30 mm.

As such, the sealing and anchoring mechanism (e.g., at least the cuff 140) can be configured to accommodate varying sizes of the aorta 101, for example, especially the varying sizes of the proximal neck 106. In some examples and as shown in FIG. 1, the cuff 140 is configured to continuously contact an inner wall of the proximal neck 106 to provide continuous sealing and anchoring at the proximal neck 106, at a part of the aorta 101 between a start of the aneurysm sac of the aneurysm 102 and the renal arteries 108a and 108b. Continuously contacting the inner wall of the proximal neck 106 refers to the fact that the cuff 140, when filled, sufficiently contacts the inner wall to form a fluid seal therewith or contacts the entire inner wall continuously, without any portion of the cuff 140 not contacting the inner wall of the proximal neck 106. The stent-like scaffold structure 145 is arranged on one end of the cuff 140. An opposite end of the cuff 140 abuts an end of the first stent graft 112.

In some examples, the cuff 140 and the first stent graft 112 are formed uniformly as a single joined part. The cuff 140 is configured to continuously contact the inner wall of the proximal neck 106, from the stent-like scaffold structure 145 to the end of the first stent graft 112. Continuously contacting the inner wall of the proximal neck 106 from the stent-like scaffold structure 145 to the end of the first stent graft 112 refers to the fact that the cuff 140, when filled, contacts the entire inner wall continuously from the stent-like scaffold structure 145 to the end of the first stent graft 112, without any portion of the cuff 140 not contacting the inner wall of the proximal neck 106 between the stent-like scaffold structure 145 to the end of the first stent graft 112. In some examples, the cuff 140, when filled, may not contact the inner wall of the proximal neck 106 all the way up to the end of the first stent graft 112. There may be a gap between the cuff 140 (when filled) and the first stent graft 112. The inflatable fill structure 130 may be inflated to fill the gap.

Some anchoring mechanisms at the proximal neck 106, between the stent-like scaffold structure 145 to the end of the first stent graft 112 or between a start of the aneurysm sac and the renal arteries 108a and 108b use two or more cuffs with lesser width than shown for the cuff 140. Making a double-cuff structure involves making a weld line in between cuff material, such that when the cuff material is filled up with polymers, two distinct cuffs are formed at the proximal neck 106, between the stent-like scaffold structure 145 to the end of the first stent graft 112 or between a start of the aneurysm sac and the renal arteries 108a and 108b, such that there would be two or more cuffs with lesser width than shown for the cuff 140.

The cuff 140 (e.g., the one, long, continuous cuff at the proximal neck 106) can be advantageous over the multi-cuff arrangements because the cuff 140 provides a larger contact surface for improved sealing and anchoring, given the increased and improved friction fit. Furthermore, the cuff material making up the cuff 140 can be expanded into a larger volume as compared to the combined volume achievable by the multi-cuff cuff arrangement. This allows improved radial expansion to accommodate different sizes (e.g., widths or diameter of the proximal neck 106. For example, when the cuff 140 is being filled with the polymer, the volume of the cuff material expands until the cuff 140 being inflated contacts the inner wall of the proximal neck 106. When there is no room along a diameter of the proximal neck 106, the cuff 140 being inflated expands longitudinally to further fill up the proximal neck 106, until the cuff 140 is entirely inflated. In various arrangements, the cuff 140 is an elongated cuff.

The cuff 140 also improves accuracy and increases a range of treatment for the entire stent graft system 100. As soon as the cuff 140 expands to a point that the cuff 140 contacts the inner wall of the proximal neck 106, it then expands longitudinally in the proximal neck 106. This allows the stent graft system 100 with the cuff 140 to be applied to a larger range of blood vessel sizes. Thus, fewer sizes for the cuff 140 are manufactured, improving flexibility and cost of product/implementation.

In some examples, the cuff 140 may be formed uniformly with the first stent graft 112. First, the second stent graft 114 with the uninflated inflatable fill structure 130 is set at the aortic bifurcation 104. Then, the first stent graft 112 with the uninflated cuff 140 is inserted into an inner lumen formed by the second stent graft 114, thus a portion of the first stent graft 112 overlaps with a portion of the second stent graft 114. Then, the inflatable fill structure 130 and the cuff 140 can be filled separately (e.g., with separate fill lines). As such, the two-piece assembly of the stent graft system 100 allows the cuff 140 to be modularly installed as an integral part of the first stent graft 112.

Other cuffs shown and described herein confer similar advantages.

FIG. 3 is diagram illustrating a stent graft system 300 with an inflatable fill structure 330 according to various arrangements. Referring to FIGS. 1, 2, and 3, the stent graft system 300 is similar to the stent graft system 100, having a stent-like scaffold structure 345 similar to the stent-like scaffold structure 145, and an inflatable fill structure 330 fillable by fill medium 332 similar to the inflatable fill structure 130 fillable by fill medium 132. The stent graft system 300 of FIG. 3 includes a single stent graft 312 instead two modular stent grafts 112 and 114 as in FIGS. 1 and 2. In other words, the inflatable fill structure 330, the cuff 340 (shown to be in an unfilled state), and the stent graft 312 form a unitary body. The inflatable fill structure 330 is provided on an exterior surface of the stent graft 312. The stent graft 312 has a bifurcated portion.

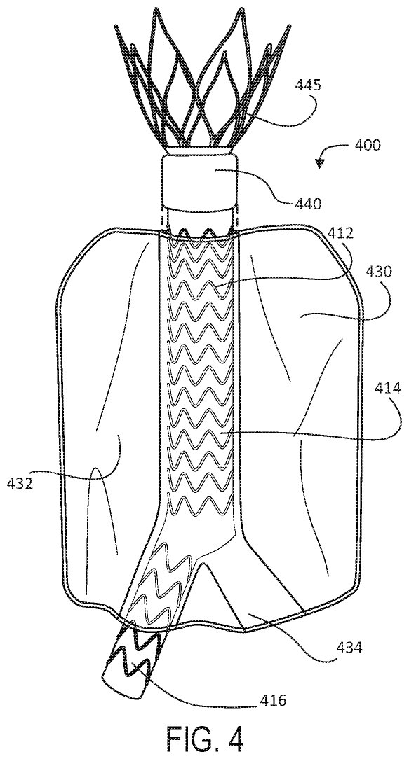

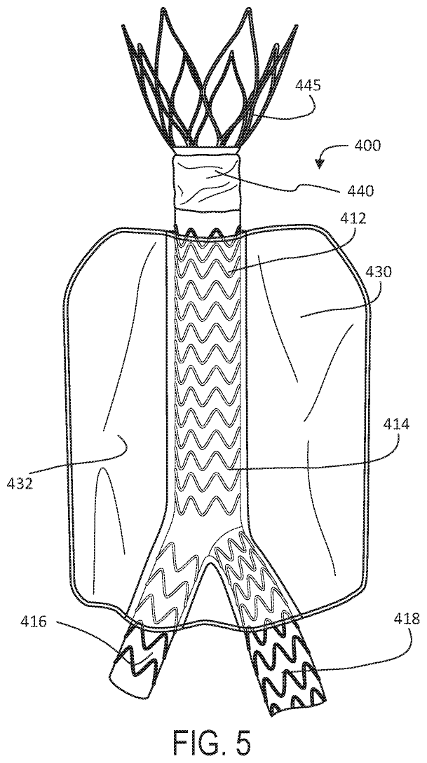

FIG. 4 is diagram illustrating a stent graft system 400 having a bifurcated inflatable fill structure 430 according to various arrangements. FIG. 5 is a diagram illustrating the stent graft system 400 of FIG. 4 with a branch stent graft 418 inserted into the bifurcated inflatable fill structure 430 according to various arrangements. Referring to FIGS. 1, 2, 3, 4, and 5, the stent graft system 400 is similar to the stent graft system 300, having a stent-like scaffold structure 445 similar to the stent-like scaffold structure 345, and an inflatable fill structure 430 fillable by fill medium 432 similar to the inflatable fill structure 330 fillable by fill medium 332.

The stent graft system 400 includes a stent graft 412. The inflatable fill structure 430, a cuff 440 (shown to be in a filled state in FIG. 4 and in an unfilled state in FIG. 5), and the stent graft 412 form a unitary body. The stent graft 412 includes a main stent graft 414 and a branch stent graft 416. In some examples, the main stent graft 414 and the branch stent graft 416 are made from a same continuous wire that extends from a limb portion (one branch of the bifurcation) to the main body portion. In other examples, the main stent graft 414 and the branch stent graft 416 are made from separate wires. The main stent graft 414 and the branch stent graft 416 form a unitary body. The stent graft 412 is placed within a cavity 434 or space of the bifurcated inflatable fill structure 430, where the cavity 434 is shaped in a bifurcated manner as shown. For example, the cavity 434 is shaped according to the shape of the main stent graft 412, the branch stent graft 416, and the inserted branch stent graft 418. In that regard, the cavity 434 includes a cavity portion for receiving the branch stent graft 418.

The branch stent graft 418 is separate from the stent graft 412, and is insertable through the portion of the cavity 434 shaped like the branch stent graft 418. Further, the stent graft 412 also includes a cavity (e.g., at where the main stent graft 414 and the branch stent graft 416 intersect) through which the branch stent graft 418 can be inserted. The bifurcated inflatable fill structure 430 therefore extends from a portion of the main stent graft 414 near or at the cuff 440 to beyond an aortic bifurcation, providing structural support for an aorta accordingly. In some examples, portions of the stent grafts (e.g., the stent grafts 112, 114, 312, 412, 414, 416, and 418) can extend and compress telescopically and include pleats in graft material allowing for the telescopic extension and compression. With reference to FIGS. 1, 4, and 5, the stent graft system 400 can be used to repair the aorta 101 in a similar manner to the stent graft system 100.

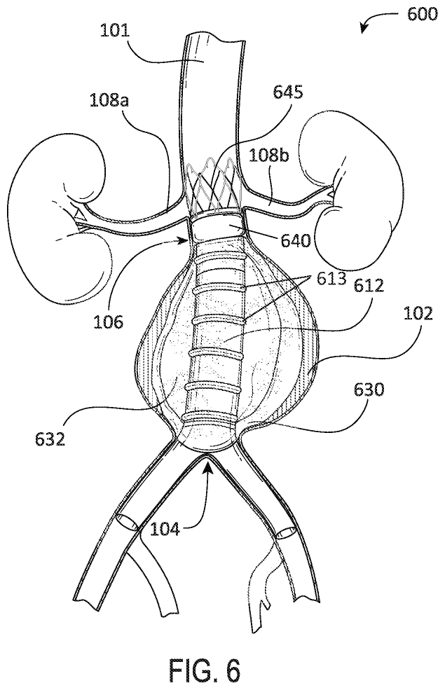

The stent graft system 400 in accordance with various arrangements includes the stent graft 412, the inflatable fill structure 430, and the cuff 440. The inflatable fill structure 430 at least partially surrounds the stent graft 412. The inflatable fill structure 430 has the cavity 434 that is bifurcated. The cuff 440 is fillable and is located outside of the inflatable fill structure 430. In various arrangements, a portion of the cavity 434 is configured to receive the branch stent graft 418 for connection to the stent graft 412. In various arrangements, the cuff 440 is located at an end of the stent graft 412. In some arrangements, the cuff 440 has a tapered shape such that it is wider at one end than at an opposite end when filled with a fill medium. In some arrangements, the stent graft system 400 can further include inflatable channels located at least partially around the stent graft 412 and surrounded by the inflatable fill structure 430. Examples of inflatable channels around a stent graft are shown in FIG. 6 and can be used in the stent graft system 400 of FIGS. 4 and 5 around the stent graft 412. In various arrangements, the cuff 440 and the inflatable fill structure 430 are separately fillable from each other to different pressures with fill medium.

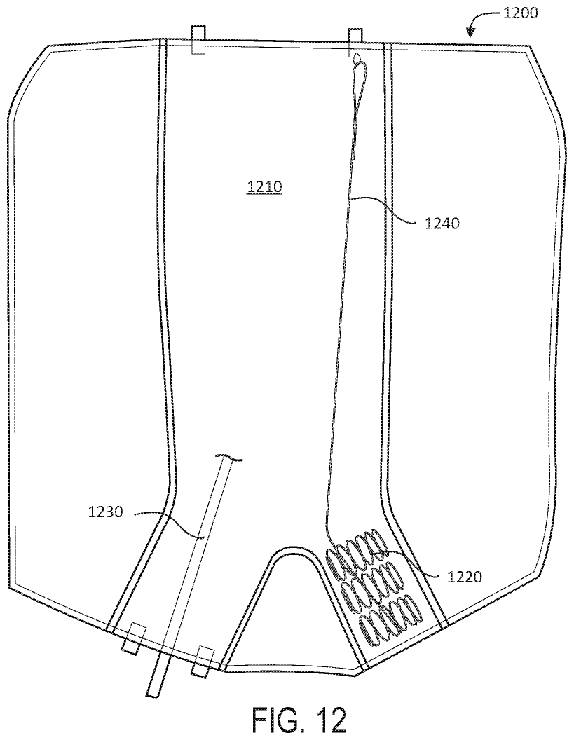

In various arrangements, the inflatable fill structure 430 is configured such that the cavity 434 that is bifurcated is longer on one side of the bifurcation than on the other side of the bifurcation. In some arrangements, a scaffold mechanism is located in the cavity 434 at least partially in an area where the branch stent graft 418 is insertable into the cavity 434, and the scaffold mechanism is configured to provide structural support to the inflatable fill structure 430 prior to the branch stent graft 418 being received within the cavity 434. Also, in some arrangements, the stent graft system 400 further includes a longitudinal support structure anchored to an end of the inflatable fill structure 430 and structurally coupled to the scaffold mechanism. Examples of the scaffold mechanism and longitudinal support structure are shown in FIG. 12 and can be used in the stent graft system 400 of FIGS. 4 and 5. In various arrangements, the stent graft 412 includes the main stent graft 414 and the branch stent graft 416 that are integrally formed, and the main stent graft 414 includes a cavity for receiving the branch stent graft 418.

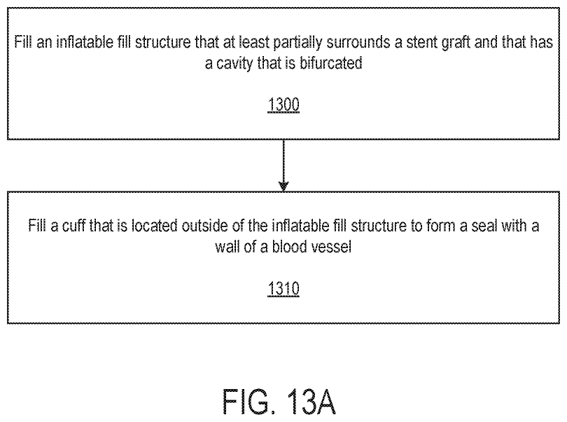

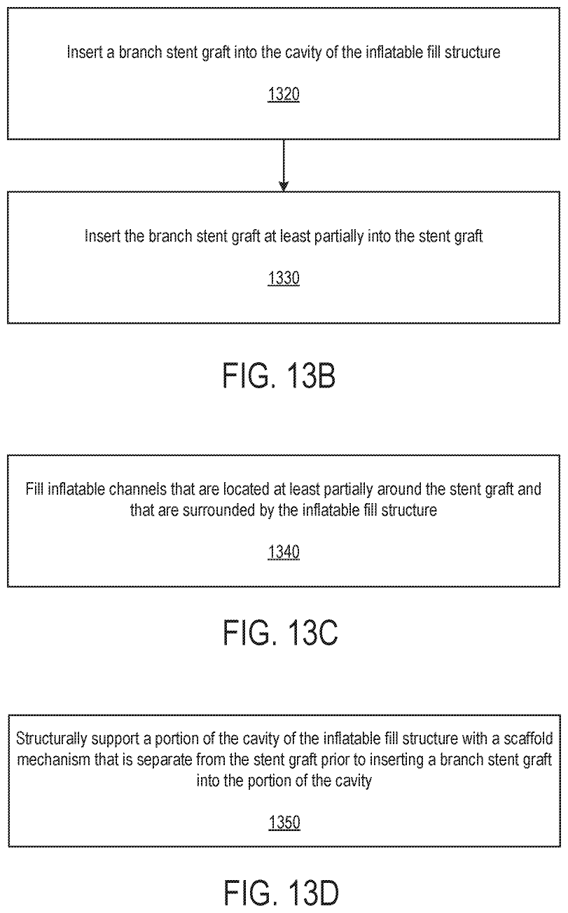

FIG. 13A is a flowchart of a method in accordance with an aspect for using the stent graft system 400 of FIGS. 4 and 5. With reference to FIGS. 1, 4, 5, and 13A, the method includes a step 1300 of filling the inflatable fill structure 430 that at least partially surrounds the stent graft 412 and that has the cavity 434 that is bifurcated, and the step 1310 of filling the cuff 440 that is located outside of the inflatable fill structure 430 to form a seal with a wall of a blood vessel, such as a wall of the proximal neck 106 of the aorta 101. FIG. 13B shows additional steps that can be used with the method of FIG. 13A. With reference to FIGS. 1, 4, 5, and 13B, in various aspects, the method further includes the step 1320 of inserting the branch stent graft 418 into the cavity 434 of the inflatable fill structure 430. Also, in various aspects, the method further includes the step 1330 of inserting the branch stent graft 418 at least partially into the stent graft 412. In some instances, the steps shown may be performed in a different order.

In various aspects, the cuff 440 and the inflatable fill structure 430 are filled to different pressures. For example, in some aspects, the cuff 440 is filled to a higher pressure than a pressure of filling of the inflatable fill structure 430. In some aspects, the cuff 440 has a size such that it extends from a bottom of renal arteries 108a and 108b to a top of the aneurysm 102 so as to form a seal in the entire proximal neck 106 region of the aorta 101 when filled. In various aspects, the method further includes filling inflatable channels that can be located at least partially around the stent graft 412 and that are surrounded by the inflatable fill structure 430.

In various arrangements, the cuff 440 has a tapered shape such that it is wider at one end than at an opposite end when filled. Also, in some arrangements, one side of the inflatable fill structure 430 that surrounds the branch stent graft 416 is longer than another side of the inflatable fill structure 430 that surrounds the branch stent graft 418. In some aspects, the method further includes structurally supporting a portion of the cavity 434 of the inflatable fill structure 430 with a scaffold mechanism that is separate from the stent graft 412 prior to inserting the branch stent graft 418 into the portion of the cavity 434. In some arrangements, the stent graft system 400 further includes a longitudinal support structure anchored to an end of the inflatable fill structure 430 and structurally coupled to the scaffold mechanism for structurally supporting the inflatable fill structure 430. Examples of the scaffold mechanism and longitudinal support structure are shown in FIG. 12 and can be used in the stent graft system 400 of FIGS. 4 and 5.

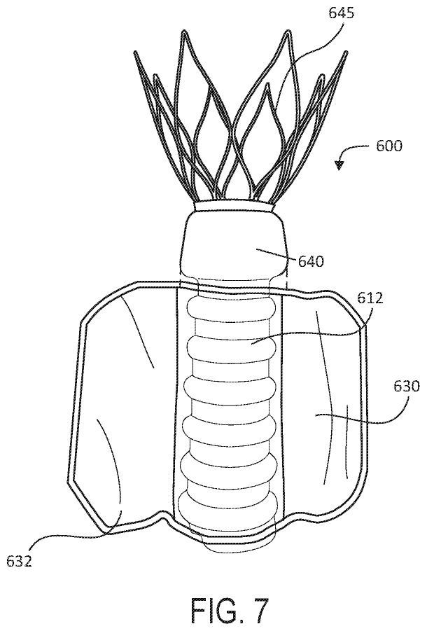

FIG. 6 is a cross-sectional view of an example of a stent graft system 600 deployed across the aneurysm 102 of the aorta 101 according to various arrangements. FIG. 7 is diagram illustrating the stent graft system 600 of FIG. 6 according to various arrangements. Referring to FIGS. 3, 6, and 7, the stent graft system 600 is similar to the stent graft system 300 shown in FIG. 3. The stent graft system 600 has a stent-like scaffold structure 645 similar to the stent-like scaffold structure 345, an inflatable fill structure 630 fillable by fill medium 632 similar to the inflatable fill structure 330 fillable by fill medium 332, and a cuff 640 similar to the cuff 340. The stent graft system 600 includes a stent graft 612 that is bifurcated. The bifurcated portion of the stent graft 612 is not shown for brevity in FIG. 7.

With reference to FIGS. 6 and 7, instead of or in addition to metal scaffolding, the stent graft 612 can be filled with polymers to provide structural integrity. In various arrangements, the stent graft 612 includes inflatable channels 613 that are fillable with polymers to provide structural integrity for the stent graft 612. The inflatable channels 613 shown in FIG. 6 can be used in the same way with the stent graft 412 of FIG. 4. With reference to FIG. 6, the inflatable fill structure 630 is fillable to fill a space in the aneurysm 102 between the aortic bifurcation 104 and the proximal neck 106. The cuff 640 is fillable to provide a seal against a wall of the proximal neck 106 between the renal arteries 108a and 108b and the aneurysm sac of the aneurysm 102.

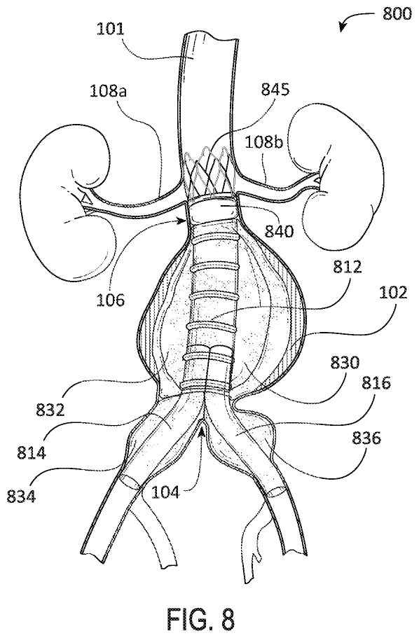

FIG. 8 is a cross-sectional view of an example of a stent graft system 800 with a main inflatable fill structure 830 deployed across the aneurysm 102, and two branch inflatable fill structures 834 and 836 deployed in iliac arteries according to various arrangements. Referring to FIG. 8, the stent graft system 800 includes a main stent graft 812 coupled to branch stent grafts 814 and 816. The main stent graft 812 is attached to the inflatable fill structure 830 that is fillable by fill medium 832. The branch stent grafts 814 and 816 are attached to the branch inflatable fill structures 834 and 836, respectively. In some arrangements, the stent grafts 812, 814, and 816 are separate from one another and include inflatable channels that are fillable with polymers using different fill lines to provide structural integrity. In some arrangements, two or more of the stent grafts 812, 814, and 816 form a unitary body to be filled with polymers using a same fill line. In some arrangements, the inflatable fill structures 830, 834, and 836 are separate from one another and are filled with polymers using different fill lines. In some arrangements, two or more of the inflatable fill structures 830, 834, and 836 form a unitary body to be filled with polymers using a same fill line.

The inflatable fill structure 830 is fillable to fill a space in the aneurysm 102 between the aortic bifurcation 104 and the proximal neck 106. The stent graft system 800 includes a cuff 840 that is fillable to provide a seal against a wall of the proximal neck 106 between the renal arteries 108a and 108b and the aneurysm sac of the aneurysm 102 in the aorta 101. The stent graft system 800 further includes a stent-like scaffold structure 845 for anchoring the stent graft system 800 in the aorta 101.

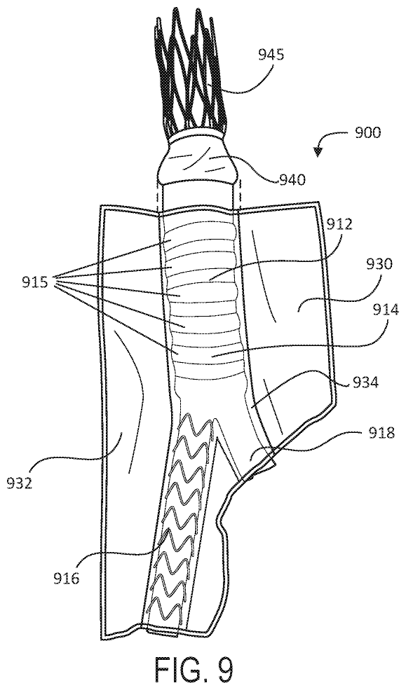

FIG. 9 is diagram illustrating a stent graft system 900 having a bifurcated inflatable fill structure 930 according to various arrangements. Referring to FIG. 9, the stent graft system 900 has a stent-like scaffold structure 945 and the inflatable fill structure 930 fillable by fill medium 932. The stent graft system 900 also includes a stent graft 912 and a cuff 940. In various arrangements, the inflatable fill structure 930, the cuff 940 (shown to be in an unfilled state), and the stent graft 912 form a unitary body. The stent graft 912 includes a main stent graft 914 and branch stent grafts 916 and 918. The main stent graft 914 and the branch stent grafts 916 and 918 form a unitary body. The stent graft 912 is placed within a cavity 934 or space of the bifurcated inflatable fill structure 930, where the cavity 934 is shaped in a bifurcated manner as shown. For example, the cavity 934 is shaped according to the shape of the main stent graft 914 and the branch stent grafts 916 and 918.

The bifurcated inflatable fill structure 930 therefore extends from a portion of the main stent graft 914 near or at the cuff 940 to beyond an aortic bifurcation, providing structural support for an aorta accordingly. The branch stent graft 916 may have metal scaffolding and may be longer than the branch stent graft 918. A portion of the inflatable fill structure 930 adjacent to the branch stent graft 916 extends from the bifurcation to conform in shape with the branch stent graft 916, and is therefore longer than a portion of the inflatable fill structure 930 adjacent to the branch stent graft 918. In various embodiments, the main stent graft 914 includes inflatable channels 915 that are fillable by a fill medium. In other examples, the branch stent graft 918 also includes inflatable channels that are fillable by a fill medium.

The method of FIG. 13A can be employed to use the stent graft system 900 of FIG. 9. With reference to FIGS. 1, 9, and 13A, the method includes the step 1300 of filling the inflatable fill structure 930 that at least partially surrounds the stent graft 912 and that has the cavity 934 that is bifurcated, and the step 1310 of filling the cuff 940 that is located outside of the inflatable fill structure 930 to form a seal with a wall of a blood vessel, such as a wall of the proximal neck 106 of the aorta 101. FIG. 13C shows an additional step that can be used with the method of FIG. 13A. With reference to FIGS. 9 and 13C, the step 1340 includes filling the inflatable channels 915 that are located at least partially around the stent graft 912 and that are surrounded by the inflatable fill structure 930.

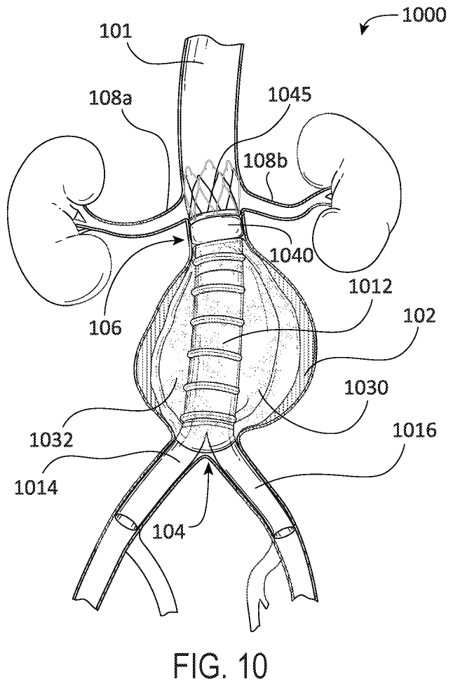

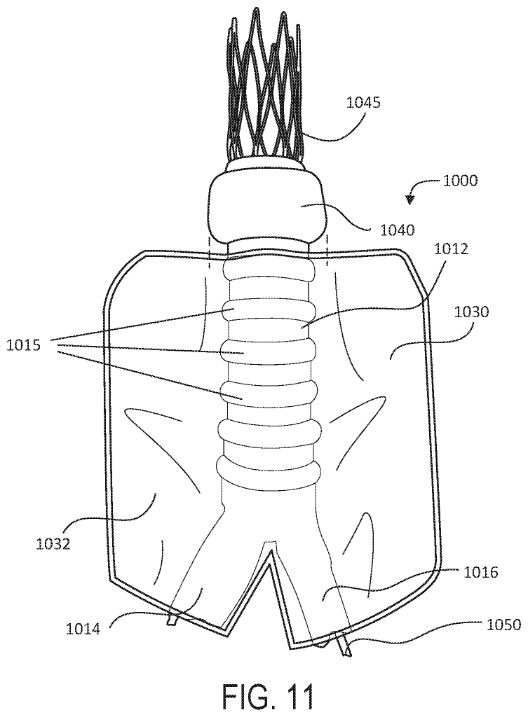

FIG. 10 is a cross-sectional view of an example of a stent graft system 1000 with a bifurcated inflatable fill structure 1030 deployed across the aneurysm 102 according to various arrangements. FIG. 11 is diagram illustrating the stent graft system 1000 of FIG. 10 having the bifurcated inflatable fill structure 1030 according to various arrangements. With reference to FIGS. 10 and 11, in various arrangements, the bifurcation portions of the bifurcated inflatable fill structure 1030 may have the same length or approximately the same length, particularly in a case where branch stent grafts 1014 and 1016 have the same length or approximately the same length. In some arrangements, the bifurcation portions of the bifurcated inflatable fill structure 1030 have the different lengths from each other. The stent graft system 1000 further includes a main stent graft 1012, inflatable channels 1015, a cuff 1040, and a stent-like scaffold structure 1045. The inflatable fill structure 1030 is fillable with a fill medium 1032.

A fill line 1050 extends through the branch stent graft 1016 and the main stent graft 1012 to allow for filling the cuff 1040 with one or more polymers. In some arrangements in which the cuff 1040 and the main stent graft 1012 are formed as a unitary body, the fill line 1050 can fill both the cuff 1040 and the inflatable channels 1015 with polymers. In some arrangements in which the cuff 1040, the main stent graft 1012, and one or both of the branch stent grafts 1014 and 1016 are formed as a unitary body, the fill line 1050 can fill inflatable channels of the unitary body with polymers.

The inflatable fill structure 1030 is fillable to fill a space in the aneurysm 102 between the aortic bifurcation 104 and the proximal neck 106. The stent graft system 1000 includes the cuff 1040 that is fillable to provide a seal against a wall of the proximal neck 106 between the renal arteries 108a and 108b and the aneurysm sac of the aneurysm 102 in the aorta 101. The stent graft system 1000 further includes the stent-like scaffold structure 1045 for anchoring the stent graft system 1000 in the aorta 101.

FIG. 12 shows a bifurcated inflatable fill structure 1200 according to various arrangements. Referring to FIG. 12, the bifurcated inflatable fill structure 1200 has a cavity 1210 through which stent grafts (as shown and described herein) can be received. The cavity 1210 is bifurcated and approximates the shape of a bifurcated stent graft (as shown and described herein). Scaffold mechanism 1220 can be provided in a branch cavity of the cavity 1210 to provide structural support before stent grafts are received in the cavity 1210. Longitudinal structural support 1240 can be anchored (e.g., hooked) to an upper portion of the inflatable fill structure 1200 adjacent to a proximal neck on one end, and structurally coupled (e.g., hooked) to the scaffold mechanism 1220, to provide longitudinal support before the stent grafts are received. A fill line 1230 can fill the inflatable fill structure 1200 and/or any inflatable channels of stent grafts and a cuff.

In various arrangements, the scaffold mechanism 1220 and longitudinal structural support 1240 are used with the bifurcated inflatable fill structure 430 of FIG. 4 in the same way that they are used with the bifurcated inflatable fill structure 1200 of FIG. 12. FIG. 13D shows a step of a method that can be used with the method of FIG. 13A. Referring to FIGS. 12 and 13D, the step 1350 includes structurally supporting a portion of the cavity 1210 of the inflatable fill structure 1200 with the scaffold mechanism 1220 that is separate from a stent graft prior to inserting a branch stent graft into the portion of the cavity 1210. That method step can also be used with the stent graft system 400 in a case where the scaffold mechanism 1220 and longitudinal structural support 1240 are used with the bifurcated inflatable fill structure 430 of FIG. 4 in the same way that they are used with the bifurcated inflatable fill structure 1200 of FIG. 12.

As shown in the figures, in various arrangements the size (e.g., diameter) of a cuff when the cuff is filled with polymer is greater than the size (e.g., diameter) of a corresponding stent graft, even if a same size of material is used for both the cuff and the stent graft body. By increasing the longitudinal dimension (along the longer dimension of the stent graft) of the cuff, the diameter of the cuff can also increase due to foreshortening. Shrinking in length along the longitudinal dimension creates slack in the cuff material diameter-wise, enabling the cuff to expand diameter-wise. Specifically, a cuff as disclosed herein may have a toroid or ring-like structure, having an inner diameter and an outer diameter. The volume of the toroid (between the inner and outer diameters) can be filled with polymer. A stent graft body as disclosed herein in various arrangements has a cylindrical shape with an axial hole (lumen), with an inner diameter defining the axial hole and an outer diameter defining the cylindrical shape. In various arrangements, a volume of the stent graft body (between the inner and outer diameters) can be filled with polymer into inflatable channels. In some examples, the difference between the inner and outer diameters of a cuff is greater than the difference between the inner and outer diameters of the stent graft body when the cuff and the stent graft body are both filled, due to foreshortening.

In addition, as shown in FIG. 7, the cuff 640 is tapered. This allows the cuff 640 to be filled up within a proximal neck that has a tapered shape. Thus, the cuff described herein can also accommodate different shapes of a proximal neck of an aneurysm.

A stent graft system in accordance with various arrangements includes a stent graft and an anchoring mechanism. The stent graft is configured to provide a lumen when the stent graft is structurally supported within a blood vessel. The anchoring mechanism is configured to provide anchoring and sealing at a proximal neck of the blood vessel. The anchoring mechanism includes a cuff made from a first inflatable material having a first inner diameter and a first outer diameter. The stent graft is made from a second inflatable material having a second inner diameter and a second outer diameter. In various arrangements, a first difference between the first inner diameter and the first outer diameter equals to a second difference between the second inner diameter and the second outer diameter when both the stent graft and the cuff are uninflated, and the first difference is greater than the second difference when both the stent graft and the cuff are inflated. In various arrangements, the cuff is filled at a first pressure, inflatable channels around the stent graft are filled at a second pressure, and the first pressure is higher than the second pressure.

A stent graft system in accordance with various arrangements includes a stent graft and an anchoring mechanism. The stent graft is configured to provide a lumen when the stent graft is structurally supported within a blood vessel. The anchoring mechanism is configured to provide anchoring and sealing at a proximal neck of the blood vessel. The anchoring mechanism includes a cuff configured to continuously contact an inner wall of the proximal neck to provide continuous sealing and anchoring at the proximal neck. In various arrangements, the anchoring mechanism includes a stent-like scaffold structure arranged on one end of the cuff, and an opposite end of the cuff abuts a first end of the stent graft. In various arrangements, the cuff is configured to continuously contact the inner wall of the proximal neck from the stent-like scaffold structure to the first end of the stent graft. In various arrangements, the stent graft includes a first stent graft and a second stent graft, the first stent graft and the second stent graft overlap to form a unified stent graft, the first stent graft abuts the opposite end of the cuff, and the first end of the unified stent graft is a first end of the first stent graft that abuts the opposite end of the cuff.

The present technology is not to be limited in terms of the particular arrangements described in this application, which are intended as illustrations of aspects of the present technology. Many modifications and variations of this present technology can be made without departing from its spirit and scope, as will be apparent to those skilled in the art. Functionally equivalent systems and methods within the scope of the present technology, in addition to those enumerated herein, will be apparent to those skilled in the art from the foregoing descriptions. Such modifications and variations are intended to fall within the scope of the present technology. It is to be understood that this present technology is not limited to particular systems and methods of using systems, which can, of course, vary. It is also to be understood that the terminology used herein is for the purpose of describing particular arrangements only, and is not intended to be limiting.

1. A stent graft system, comprising:
a stent graft;
an inflatable fill structure at least partially surrounding the stent graft, the inflatable fill structure having a cavity that is bifurcated; and
a cuff that is fillable and that is located outside of the inflatable fill structure.

2. The stent graft system of claim 1,
wherein a portion of the cavity is configured to receive a branch stent graft for connection to the stent graft.

3. The stent graft system of claim 1,
wherein the cuff is located at an end of the stent graft.

4. The stent graft system of claim 1,
wherein the cuff has a tapered shape such that it is wider at one end than at an opposite end when filled with a fill medium.

5. The stent graft system of claim 1, further comprising:
inflatable channels located at least partially around the stent graft and surrounded by the inflatable fill structure.

6. The stent graft system of claim 1,
wherein the cuff and the inflatable fill structure are separately fillable from each other to different pressures with fill medium.

7. The stent graft system of claim 1,
wherein the inflatable fill structure is configured such that the cavity that is bifurcated is longer on one side of the bifurcation than on the other side of the bifurcation.

8. The stent graft system of claim 1, further comprising:
a scaffold mechanism located in the cavity at least partially in an area where a branch stent graft is insertable into the cavity;
wherein the scaffold mechanism is configured to provide structural support to the inflatable fill structure prior to the branch stent graft being received within the cavity.

9. The stent graft system of claim 1, further comprising:
a longitudinal support structure anchored to an end of the inflatable fill structure and structurally coupled to the scaffold mechanism.

10. The stent graft system of claim 1,
wherein the stent graft includes a main stent graft and a branch stent graft that are integrally formed, and the main stent graft includes a cavity for receiving a second branch stent graft.

11. A method of using a stent graft system, the stent graft system including a stent graft, an inflatable fill structure, and a cuff, the method comprising:
filling the inflatable fill structure that at least partially surrounds the stent graft and that has a cavity that is bifurcated; and
filling the cuff that is located outside of the inflatable fill structure to form a seal with a wall of a blood vessel.

12.-20. (canceled)

21. A stent graft system, comprising:
a stent graft;
an inflatable fill structure at least partially surrounding the stent graft, the at least one inflatable fill structure having at least one cavity;
a cuff that is fillable and that is located outside of the inflatable fill structure.

22. The stent graft system of claim 21, wherein the inflatable fill structure comprises multiple discrete cavities.

23. The stent graft system of claim 21, wherein the inflatable fill structure comprises a branched cavity.

24. The stent graft system of claim 21, wherein the inflatable fill structure comprises cavities configured to provide access to multiple arteries.

25. The stent graft system of claim 21, wherein the inflatable fill structure comprises cavities configured for fluid communication with various arteries such as iliac and renal arteries.

26. The stent graft system of claim 21, comprising a fenestrated stent graft.

27. The stent graft system of claim 21, comprising branch stent grafts anchored to iliac and renal arteries.

28. The stent graft system of claim 21, comprising a plurality of inflatable fill structures.

29. The stent graft structure of claim 26, further comprising a branch stent graft configured to be inserted into the fenestration.
